(19) United States
(12) Patent Application Publication
(10) Pub. No.: US 2004/0059017 A1
(43) Pub. Date: Mar. 25, 2004

(54) Method for producing a partial or complete active ingredient coating on and in implants and onplants

(76) Inventors: Joerg Bauer, Darmstadt (DE); Uwe Emil Christian Grabuer, Wiesbaden (DE)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/433,706

(22) PCT Filed: Dec. 4, 2001
(86) PCT No.: PCT/EP01/14157

(30) Foreign Application Priority Data
Dec. 4, 2000 (DE) 100 60 354.8

(51) Int. Cl. A61L 2/00

(52) U.S. Cl. 523/102

(57) ABSTRACT

The invention relates to a method for producing a partial or complete active ingredient coating on and in implants and onplants. The aim of the invention is to allow local therapy at the site of action of an article (1) on or in the living body. To this end, the invention provides for a device with which an article (1) can be coated with an active ingredient. Said device comprises a first element that keeps ready at least one active ingredient, and another element for applying the active ingredient to the article (1). The device further comprises an irradiation device (3) whose energetic radiation is directed to the applied active ingredient so as to directly coat the article (1) with the active ingredient.

### BACKGROUND INFORMATION

The present invention relates to a method and a device for coating an object with an active substance.

Medical technology has recorded astounding successes in recent decades. This has resulted from both differentiated treatment methods and medical equipment having evidently achieved a much higher quality in medical institutions—clinics, university facilities—and/or in outpatient treatment. Further improvements may thus frequently only be achieved through highly innovative instruments or methods.

In the present case, through the novel coating methods according to the present invention, finished products or semi-finished products of the product group of medical products or drugs or similar products may be supplemented in such a way that the effect is no longer used exclusively for the original intended purpose, but rather has the additional function of local introduction of an active substance: in this way, effects may be achieved which are used, for example, to reduce the potential for allergic reaction and inflammation or to minimize immunological processes, to encourage regenerative processes and resistance to infection, and to combat contamination, or which occur in combination with one another.

Due to the manifold possibilities, in the following, the method of the present invention is to be illustrated using the example of infections. However, this is only an example for all other effects.

Postoperative infections may be reduced to infection rates around 1% through the standardization of the operating methods and materials used during standard operations. A large part of these infections may not be corrected by the defense mechanisms of the body. For this purpose, the organism requires antibiotics, which are normally given before, during, and after the operation due to the unforeseeable factors.

With the growth of spare-part surgery, a new dimension in the quality of infection risks has now arisen. An implant or onplant introduced through a body opening may harbor infection risks directly on its surface, which, once implanted, receive an ideal culture medium for their expansion. Even the microbiological states in the aseptic operating rooms, to be considered optimum in the meantime, at least in the larger industrial countries, do not diminish a certain risk of infection caused by the operation per se, the material used, and the people involved.

For these reasons, various methods are used for infection prophylaxis. Thus, for example, antibacterial active substances are injected and/or supplied via the stomach or the bloodstream. However, the antibacterial active substances are typically distributed in a wide area in the organism and not at the location where they are needed. In addition, generally distributed antibiotics have the great disadvantage that they produce undesired and often harmful side effects.

It is thus the goal of research and development to develop novel antibiotic applications which allow the active substance to be placed where it is needed and limit the dosing of the locally applied active substance to a safe minimum amount.

One direction is the development of carrier materials which release an active substance on location. This release typically occurs over a certain period of time with different mass distributions over the release period. In this case, auxiliary materials, to which the active substance is bound, are also implanted in the operation wounds, in order to unfold their effect there, i.e., the combating and prevention of infections.

Those products in which an implant is connected through the body to the outside world, such as with external fixators, are to be integrated into a further group of infections. In this case, the location of infection is usually the point of passage through the skin of the patient. Even strong and frequent hygiene measures often do not help here, so that additional therapy must be performed.

Considered as a whole, measures for combating infections and prophylaxis against infections are a must according to the current state of medicine. Since the current methods contain the danger of undesired side effects, the development of methods of localized active substance administration is a technological challenge.

According to the present invention, a method has now been developed, using which the disadvantages of the possible therapies described above are remedied, and a local therapy may be performed at the target location, without introducing secondary implants or requiring an additional measure such as injections or tablets. At the same time, the use of such materials coated according to the present invention is possible in inpatient or outpatient treatment.

The object is achieved according to the present invention in that an active substance is applied to the inner and/or outer surface of an implant to be implanted or an onplant to be onplanted in such a way that the surface provided with active substance is provided essentially completely with this active substance in a completely closed coating, or partially using spacings relevant for the active substance, which are a function of the diffusion behavior of the active substance. In this case, the active substance may cover only segments of the surface on the part or may enclose it completely. For segmented coating, single-sided or multisided coating is possible according to the present invention, this coating also being able to be closed or segmented. According to the present invention, the active substance coating is alternately a hard coating, an elastic coating, or a soft coating, graduation and/or layering of different viscosities of the active substance coating representing variations according to the present invention.

In addition to this hardness classification, there is the possibility of applying different active substances at different points of the part, so that according to the present invention a coating may be produced having locally different active substances, or combinations which differ both in hardness and in composition may be produced from one or more active substances.

According to the present invention, a coating having active substance may also be produced which includes a graduated release rate of the active substance(s), so that in the coating method according to the present invention, a large variance results, having many degrees of freedom in regard to design. The strength of the active substance on the surface of the part is essential in this case. Through the part coated according to the present invention, the active substance is inserted into the wound or laid on it, without significant quantities of the active substance able to fall outside the target location through friction or abrasion. In the classic case, for example, metallic endoprostheses are driven into the prepared bone bed using a striking tool. The resulting press fit is essential for the bone to grow onto the endoprosthesis.

If bacteria are introduced into the wound through contamination of the prosthesis or of the tool or other surgical equipment, the implant and the bone tissue are unprotected. The operation may certainly last up to an hour or more. The danger of additional introduction of bacteria grows because of this. If the endoprosthesis is now provided with a prophylactic protection, a source of infection which is introduced may be combated directly on location and the unhindered healing of the implant may be ensured. Loosening of prostheses due to infected areas having elevated cell activities may no longer occur.

In addition, a not insignificant abrasive friction arises on the surface of the implant during the introduction of the prosthesis into the bone bed, which makes solid adhesion of the active substance onto the implant surface necessary, because the prosthesis tip or its edges and curves must or may also be prophylactically protected.

In the case of plating of a defective point, the related art is to lay a metallic plate so it bridges the defect and to fix it using special screws. In this case, the regions which are most endangered are the bottom of the plate and the cavities resulting due to the screws. If these regions may now be provided with a prophylactic active substance coating, the danger of loosening is reduced, and the positive course of convalescence increases with the reduction of the loosening rate.

In a further requirement, implants project through the skin to the outside and fulfill their function in their inner and outer design. This is the case above all for fixators, for Kirschner wires, and for stretching and expansion devices, among other things. In this case, the greatest point of danger, besides the danger of infection through the operation itself, is infection through the point of passage, which may travel along the implant channel into the wound. A hard protection through an active substance represents a significantly better product for the patient in this case than the current medicinal treatments.

These possible uses, prostheses, and examples of implants described above, which are to be understood as examples, share the feature of elimination of the possibility of error through displacement of the active substance and reduction of the danger of human errors through underdosing and/or overdosing.

These models may be transferred to all metallic implants, onplants, and, according to the present invention, also to the instruments used. The instruments are still of secondary significance, but with the increase in highly sensitive operations or for operations without optimum operating room environments, such as in emergency operations at accident scenes or in countries having lower hygienic standards, the instruments will certainly be of increasing importance in the coming years.

The passive protection of instruments against colonization through a coating of this type may also be advisable if, for example, emergency surgical instruments are to survive long storage times.

Notwithstanding this, in a further application area, materials other than metallic materials are to be considered. Thus, the protection of patients from infections is certainly also a function of the purity of the secondary auxiliary materials, although this is not as significant as in implants. The protection using an active substance may at least reduce possible dangers to the peripheral regions and to parts visible to the patient.

The healing of an incision which was subject to bandaging is certainly not critical. However, the scar is typically less “beautiful” in the final effect, i.e., more bulging and pronounced in the scar region, upon the occurrence of an inflammation than without inflammation. If, according to the present invention, the closing means of such a wound, whether it is caused by an intervention or is present as a laceration, or originated otherwise, is produced using a material protected by an active substance coating according to the present invention, the probability of infection may thus be reduced. Above all, surgical sutures and the needles used, the clamps of clamping devices and other objects are considered as materials here.

Inflammations occur especially frequently through the use of injection devices. This problem has particularly entered discussion and awareness due to the transmission of AIDS. However, this problem may not be ignored even in crisis areas or in the event of environmental catastrophes. If these injection needles are now coated with a prophylactic active substance protection in, for example, a hard variant, this problem may certainly be reduced in many cases.

If, in a special application, devices are temporarily used, their temporary protection is also possible. In this special case, for example, the wearers of hearing aids are considered, who often have to deal with inflammatory reactions at the contact points in the ear. If the region of contact of such a hearing aid is now provided with an active substance protection which has a certain material hardness for such a case, in certain circumstances the ointments, which clog up the device and sometimes also reduce the hearing quality, may be replaced. The concentrated treatment of the inflammation is now also possible while the hearing aid is worn.

In a further case, the coating of vision aids is provided according to the present invention. In this case, the supporting components of the vision aid in the nasal region and the ear pieces over the ears in particular are coated with an active substance coating, so that even if there is an existing inflammation, treatment is possible without having to dispense with the vision aid.

The prophylactic supply of active components with an active substance may represent a further application according to the present invention. Thus, protection of pumps, metering devices, signaling devices, pulse generators, or analytic implants such as insulin detectors and other things is completely realistic.

In more recent practice, however, dental implants have also represented a suitable method partner. Thus, a jaw implant or dental implant which is coated with an active substance of this type is much less endangered than one which is unprotected.

If one considers the methods of the operations and the materials used in this new medical field, the use of synthetic tendons and ligaments and cartilage replacement is a rapidly growing application marketplace of greatly increasing significance. According to the present invention, these materials may now also be provided with an active substance protection using this method. In this case, the active substance may be applied only onto the outer surface, or may also be placed inside the parts. Through the special processing method, the active substance may be tailored to the movability of the part in this case, so that no functional restriction may occur.

In particular, the possibilities of active substance coating by the method are suitable for obtaining specific workpiece properties in regard to porosity, flexibility, surface structure, and the like. Since the coating is performed without the aid of carrier substances, the volume applied for releasing a quantity of active substance is always smaller than previously known, so that the implanted foreign volume is minimal. Foreign substance reactions due to the active substance are therefore always minimized. This is not the case for carrier-bound active substances, which are always subject to the problem of foreign substance reactions. Additional cell activity, caused by the carrier substances or their degradation products, is also not caused by monomaterial administration. Due to the reduction of the additional cell activities, the effectiveness of the active substance is not overshadowed, so that it may unfold its optimum effect. The problem of gap formation may also be reduced as a result of the carrier-free active substance coating. Normally, the coating thickness of the carrier increases, which may cause significant spacing between the workpiece part and, for example, bones. This spacing forms a gap between the part and the body part as the carrier dissolves while the active substance is released simultaneously, which causes undesired mechanical, biomechanical, and biological instabilities. If this gap is minimized, the danger of contact play (movement) is automatically reduced. In particular, the possibility is provided through the method according to the present invention of introducing a very high dose of active substance in a minimum coating thickness. These coating thicknesses may certainly be in the range below the scale of μm, so that active substance coating thicknesses in the nm range may be implemented.

The method itself is achieved according to the present invention in that the part is treated at low temperature using energy beams. This method is necessary according to the present invention since the active substances to be used are typically extremely temperature-dependent. Overall, the method of producing a stable active substance coating may be achieved if a material is prepared by treating its microsurface structure in such a way that it absorbs liquids and/or water over the entire surface without agglomerations resulting due to the surface tension. As a result of the uniform wetting of the part surface, metallic implants or onplants in this case, a correspondingly uniform distribution of the active substance(s) in the moisture film results. Elevations of the liquid density at the edges may be ignored, since the resulting active substance coating only reaches a fraction of the thickness of the liquid coating.

The special feature of the method according to the present invention is that the beams supplied only cause heating of the liquid coating to a small extent. The supply of energy beams has been shown to be especially advantageous for the method according to the present invention, since the effect of the beams on the active substances themselves is very slight. Only the amount of solvent necessary for application of the active substance must be eliminated, formation of a crystalline layer structure of the active substance occurring simultaneously. Depending on the length and intensity of the supply of the electromagnetic energy, a harder or softer crystalline product of pure active substance is obtained. Due to the method, one or more further coatings of the same active substance, which are made softer by using a lower dose of energy or are made harder through a higher dose, may be applied after production of a first coating, or the next application may be performed using another active substance or mixtures of active substances, which may also be implemented as being harder or softer. If this sequence is practiced, different active substances—or identical active substances—may be released by or from of the implant/onplant surface one after another or at different dissolution rates. In this way, controlled supply of the wound area with the active substance(s) is possible.

However, it is also essential according to the present invention that the active substances may not be dissolved spontaneously, as is normal for most active substances in their natural consistency, but rather dissolve over a period of time.

In the following, an exemplary embodiment is described which clarifies both the method and its possibilities for exemplary purposes.

A hip joint endoprosthesis made of a cobalt-chromium-molybdenum steel with a standard head connection having a Eurocone, whose lowermost prosthesis tip is polished, is covered with a temporary film after basic cleaning at the tip. The collar of the prosthesis and the cone itself, but without the prosthesis neck, are also covered with a temporary film. The remaining exposed surfaces are now microsurface textured according to conventional methods, for example, using glass bead blasting, etching, grinding, or other surface treatment methods. In this case, special value is placed on the chemical purity of the blasting agent or the treatment agent in order to avoid surface impurities. After the removal of the cover films, basic cleaning of the hip joint endoprosthesis is performed in order to remove possible blasting agent residues or treatment agent residues. This is typically performed using brushes and ultrasound baths. Subsequently, the hip joint endoprosthesis is completely degreased and the degreasing agent is removed without residue. The hip joint endoprosthesis is placed in a holder for further processing and sent under monitored conditions to a suitable location for post-treatment.

The surface area which is intended for the coating was determined mathematically. For this application, a coating surface of precisely 80.10 cm resulted.

In a simultaneous work cycle, the active substance is prepared for the coating. After appropriate active substance assays for purity and activity, and other quality control and safety assays, a reactant batch is produced. For this purpose, a precisely weighed quantity of active substance is added to a supply of solvent, double-distilled sterile water in this case, and homogeneously dissolved or suspended until the active substance is completely dissolved or suspended. In our example, this is precisely 50.000 grams gentamicin sulfate, whose activity was determined to be 95%, in 100.000 grams water. The resulting solution is provided for further treatment at a specific temperature—in this case at 15° C. An active substance solution having a weight of 150 grams results, of which 50.000 grams represent gentamicin sulfate, representing an active quantity of 47.500 grams active substance.

In the next work cycle, the solution of the active substance is applied uniformly onto the surfaces of the hip joint endoprosthesis to be treated. These are the prosthesis shaft without its tip (the supply of active substance occurs in this case through a marrow cavity plug, which has also been treated using this method according to the present invention, lying in front of the prosthesis tip), the prosthesis collar on its bottom side, and the prosthesis neck without the adjoining cone. The application is performed by a suitable device using defined quantities.

In this application, the following balances result: Weight of the hip joint endoprosthesis before the coating 180.500 grams. After the application of the coating this prosthesis has a weight of 181.900 grams. Therefore, an application of active substance solution of 1.400 grams results. Of these 1.400 grams of active substance solution applied, 0.4666 grams are gentamicin sulfate, of which 0.4433 grams represent active substance. The remaining quantity is represented by 0.9333 grams water.

In the next work step, the hip joint endoprosthesis thus prepared using the active substance solution is subjected to a dose of electromagnetic radiation in a suitable holder. The radiation is applied using a frequency or frequencies which preferably lie in the following frequency bands: L-band (0.39 GHz to 1.55 GHz), S-band (1.55 GHz to 3.9 GHz), C-band (4 GHz to 6 GHz), X-band (6.2 GHz to 10.9 GHz), K-band (10.9 GHz to 36 GHz), Q-band (36 GHz to 46 GHz) or V-band (46 GHz to 56 GHz), and over the V-band. The frequencies at which absorption in water is increased are suitable for the coating in particular. The energy is preferably supplied intermittently or bundled.

The energy portion accounting for the active substance of the hip joint implant only amounts to a slight power output in this application.

After the fixed reaction time has elapsed, the now coated hip joint implant is removed from the reaction chamber.

The material balance now results in a weight of 180.9610 grams, of which 180.5 grams are the hip joint endoprosthesis and 0.4610 grams are the quantity of active substance. The 0.0056 grams quantity differential are standardized quantities lost due to contact points and evaporation. The effective quantity applied to the hip joint endoprosthesis is accordingly 0.4379 grams active substance.

Converted to the calculated coating surface area, an active substance quantity of 0.00547 g/cm² coated area results.

The release experiments performed in physiological table salt solution/water/serum resulted in an exponentially falling release quantity over time. This quantity was far above the minimum concentration in the initial starting phase of the release. A concentration gradient over the surroundings could not be determined. Even after long storage times in the elution media, significant quantities of active substance were still found by analysis. If one now considers all examples of implants or onplants or peripheral parts listed, a whole array of further fields of application and materials, which could be processed using this method, would be conceivable to one skilled in the art and derived from the examples illustrated. These other applications, which are not described in greater detail, are also claimed according to the present invention.

The fields of active substances which could be processed using this method include above all the active substances of gentamicin, clindamycin, vancomycin, penicillins, and comparable or similar materials, such as weight reducing agents/appetite suppressants, analeptics/antihypoxemics, analgesics/antirheumatics, antiallergics, antianemics, antiarrhythmics, antibiotics/antiinfectants, antidementia drugs (nootropics), antidiabetics, antidotes, antiemetics/antivertigo drugs, antiepileptics, antihemorrhagics (antifibrinolytics and other hemostatics), antihypertensives, antihypoglycemics, antihypotensives, anticoagulants, antimycotics, antiparasitic agents (external), antiphlogistics, antitussives/expectorants, arteriosclerosis agents, balneotherapeutics and agents for heat therapy, beta receptor blockers, calcium channel blockers and inhibitors of the renin-angiotensin system, bronchiolytics/antiasthmatics, cholagogues and gallbladder pharmaceuticals, cholinergics, corticoids (internal), dermatics, disinfectants/antiseptics, dietetic agents/nutrition treatments, diagnostics and agents for diagnosis preparation, diuretics, circulatory enhancement agents, withdrawal agents, enzyme inhibitors, enzyme preparations and transport proteins, fibrinolytics, geriatric agents, gout agents, influenzal agents and agents against colds, gynecological agents, hepatic agents, hypnotics/sedatives, pituitary hormones, hypothalamus hormones, other regulatory peptides and their inhibitors, immune modulators, infusion and standard injection solutions, organ perfusion solutions, cardiacs, caries agents, periodontosis agents and other dental preparations, coronary agents, laxatives, antilipemics, local anesthetics/neurotherapeutics, gastrointestinal agents, migraine agents, mineral preparations, oropharyngeal treatments, muscle relaxants, narcotics, neuropathy preparations and other neurotropic agents, ophthalmics, osteoporosis agents/calcium metabolism regulators, otologics, Parkinson's agents and other agents against extrapyramidal disorders, psychopharmaceuticals, rhinologic agents/sinusitis agents, reconstituents/tonics, thyroid treatments, serums, immunoglobulin and vaccines, sexual hormones and their inhibitors, spasmolytics, thrombocyte aggregation inhibitors, tuberculosis agents, immune enhancers, urologics, venous treatments, vitamins, wound treatment agents, cytostatics, other antineoplastic agents and protectants, prepared serums/registered homeopathics, biomaterials/medical plastics/miscellany. Mixtures of these individual materials with one another may significantly expand the effective spectrum and thus prevent multiple possible infections. However, the method is also suitable for the purpose of using the active substance coating primarily provided for infection protection as a carrier. Thus, for example, active substances for combating illnesses may be added to the anti-inflammatory active substance, which forms the hard active substance coating. These include, for example, cytostatics for actively combating proliferative cells. In particular and according to the present invention, materials which are essentially suitable for influencing specific biological procedures may also be added or attached into or onto the active substance coating. In the case of bone binding of implants, these are particularly materials such as BMP (bone morphogenic proteins) or FGF (fibrogen growth factors) or genetically manipulated or produced materials which positively influence bone growth. This addition/incorporation of materials of this type without the use of carrier materials has the special character according to the present invention of substance introduction which is bound to the active substance. The free ability to unfold the effect of the substances which intervene in the cells without the obstructive effect of the secondary carrier materials is to be emphasized in this case, as is the active substance protection caused by the actual prophylactic active substance coating, which also protects the materials such as the growth factors (e.g., BMP, FGF) and the like from infection colonization.

Surprisingly, and therefore according to the present invention, it has been shown that the method is also suitable for coating bone replacement materials. Thus, for example, the same hard, graduated, single-layer, or multilayer active substance coating may be applied during the coating of porous ceramic implants. The known spontaneous release of active substances, particularly antibiotics, from such materials may be significantly improved, so that the effect of the method is the same as it was on the surface of prostheses. In particular, bone replacement materials based on calcium and phosphate, calcium and carbonate, and calcium and sulfate of natural or synthetic origin may be processed. In this case, both materials of ceramic composition and materials having organic components may be successfully coated.

The same is also shown, and is claimed according to the present invention, for the coating of plastics, such as for inlays for prostheses, plates, washers, drains, syringe parts, wedges, gauzes, and skins. From the manifold materials coated, it may be concluded that other materials could also be coated using these methods. Therefore, the coating of materials such as glass ceramic, glass, metal ceramic, ceramic, and the transition materials are claimed according to the present invention. Finally, the method may also be used for coating jewelry parts (earrings, piercing parts, etc.).

The present invention is to be described in greater detail in the following on the basis of preferred exemplary embodiments and with reference to the attached drawing, identical reference numbers referring to identical or similar components in the individual figures of the drawing.

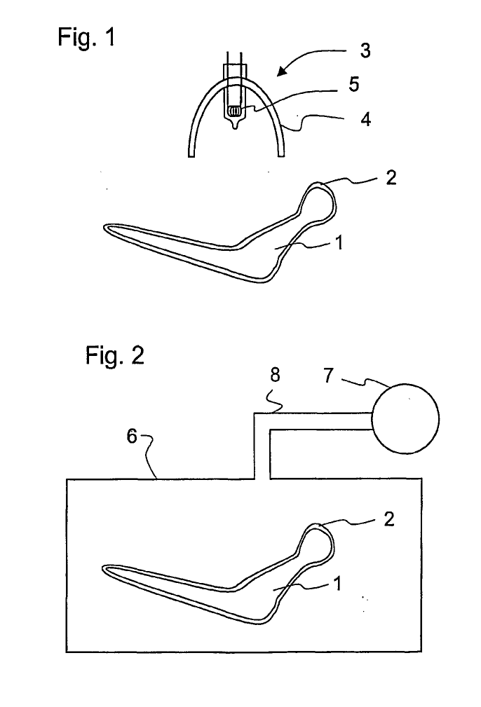

FIG. 1 shows a first embodiment of a device for coating an object with an active substance according to the present invention, and

FIG. 2 shows a further embodiment of the device for coating an object with an active substance according to the present invention.

FIG. 1 shows a first embodiment of a device for coating with an active substance. The objects are medical implants or onplants in particular. Implant 1 shown in FIG. 1 is a hip joint implant, for example. First, using a device (not shown), implant 1 is provided with a coating which contains one or more active substances. In this case, the coating may be a solution of the active substances in a suitable solvent, such as water or alcohol. The implant treated in this way is subsequently irradiated with electromagnetic waves using a radiation source 3. In the case of the embodiment shown in FIG. 1, the radiation source is a light source which includes a radiation generator in the form of a coiled filament 5. The radiation emitted by the coiled filament impinges onto implant 1, which is coated with coating 2, both directly and reflected via reflector 4.

Reflector 4 may be designed for the purpose of influencing the radiation spectrum emitted by filament 5. For example, reflector 4 may be designed as a cold-light reflector through coating using a suitable interference coating, so that particularly the shortwave components impinge on the implant.

A further embodiment of a device for coating with an active substance is shown in FIG. 2. In this case, implant 1, which is coated with a coating 2 made of active substances dissolved in a solution, is introduced into a metallic conductive housing 6, into which, for example, microwaves are radiated via a waveguide 8 for treating the implant, and/or for manufacturing the active substance coating. The electromagnetic waves are generated by a source 7. For example, an electromagnetic generator may be used as source 7.

Through the treatment using the radiation sources of the exemplary embodiments described on the basis of FIGS. 1 and 2 of devices for coating implants, an active substance coating is finally produced without secondary carrier materials on the implant.

What is claimed is:

1. A device for coating an object with an active substance, comprising:
a first device, which keeps at least one active substance ready for use,
a further device for applying the active substance onto the object, and
a radiation device, whose energy radiation is directed onto the active substance applied in order to coat the object directly with the active substance.

2. The device as recited in claim 1,
wherein the radiation device produces electromagnetic radiation.

3. The device as recited in one of claims 1 or 2,
wherein the radiation device produces radiation having identical or different forms, in particular intermittently or bundled.

4. The device as recited in one of the preceding claims,
wherein the first device keeps a mixture of at least two active substances ready for use.

5. The device as recited in one of the preceding claims,
wherein the first device keeps a mixture of at least one active substance or an active substance mixture as recited in claim 4 and another material ready for use, in particular in a solution or suspension.

6. A mixture for a device as recited in claim 5,
wherein the other material is water or alcohol, in particular double-distilled sterile water.

7. The device as recited in one of claims 4 through 6,
wherein the further device is used for applying the active substance mixture or the mixture.

8. The device as recited in one of the preceding claims,
wherein the active substance includes one of the following substances or a combination of the following substances:
weight reducing agents/appetite suppressants, analeptics/antihypoxemics, analgesics/antirheumatics, antiallergics, antianemics, antiarrhythmics, antibiotics/antiinfectants, antidementia drugs (nootropics), antidiabetics, antidotes, antiemetics/antivertigo drugs, antiepileptics, antihemorrhagics (antifibrinolytics and other hemostatics), antihypertensives, antihypoglycemics, antihypotensives, anticoagulants, antimycotics, antiparasitic agents (external), antiphlogistics, antitussives/expectorants, arteriosclerosis agents, balneotherapeutics and agents for heat therapy, beta receptor blockers, calcium channel blockers and inhibitors of the renin-angiotensin system, bronchiolytics/antiasthmatics, cholagogues and gallbladder pharmaceuticals, cholinergics, corticoids (internal), dermatics, disinfectants/antiseptics, dietetic agents/nutrition treatments, diagnostics and agents for diagnosis preparation, diuretics, circulatory enhancement agents, withdrawal agents, enzyme inhibitors, enzyme preparations and transport proteins, fibrinolytics, geriatric agents, gout agents, anti-influenza agents and agents against colds, gynecological agents, hepatic agents, hypnotics/sedatives, pituitary hormones, hypothalamus hormones, other regulatory peptides and their inhibitors, immune modulators, infusion and standard injection solutions, organ perfusion solutions, cardiac drugs, caries agents, periodontosis agents and other dental preparations, coronary agents, laxatives, antilipemics, local anesthetics/neurotherapeutics, gastrointestinal agents, migraine agents, mineral preparations, oropharyngeal treatments, muscle relaxants, narcotics, neuropathy preparations and other neurotropic agents, ophthalmics, osteoporosis agents/calcium metabolism regulators, otologics, Parkinson's agents and other agents against extrapyramidal disorders, psychopharmaceuticals, rhinologic agents/sinusitis agents, reconstituents/tonics, thyroid treatments, serums, immunoglobulin and vaccines, sexual hormones and their inhibitors, spasmolytics, thrombocyte aggregation inhibitors, tuberculosis agents, immune enhancers, urologics, venous treatments, vitamins, wound treatment agents, cytostatics, other antineoplastic agents and protectants, prepared serums/registered homeopathics, biomaterials/medical plastics/miscellany, or growth factors.

9. A method of coating an object with an active substance, including:
a) applying at least one active substance to the object and
b) irradiating the active substance applied in order to coat the object directly without secondary carrier materials.

10. A method of coating an object with an active substance,
wherein
a) at least one active substance is applied to the object using secondary carrier material and
b) the active substance applied using secondary carrier material is irradiated.

11. The method as recited in claim 9 or 10,
wherein the active substance includes one of the substances described in claim 8 or a combination of these substances.

12. The method as recited in one of claims 9 through 11,
wherein a mixture of at least two active substances is applied to the object.

13. The method as recited in one of claims 9 through 12,
wherein the active substance or the active substance mixture as recited in claim 11 is mixed with another material to form a solution or suspension.

14. The method as recited in claim 13,
wherein the other material, alcohol or water or another solvent, in particular double-distilled water, is present temporarily.

15. The method as recited in one of claims 13 or 14,
wherein the solution or suspension or emulsion is applied to the object.

16. The method as recited in one of claims 9 through 15,
wherein the active substance mixture or the solution or the suspension applied is irradiated.

17. The method as recited in one of claims 9 through 16,
wherein the irradiation is performed through energy radiation.

18. The method as recited in one of claims 9 through 17,
wherein the irradiation is performed through electromagnetic radiation.

19. The method as recited in one of claims 9 through 18,
wherein the irradiation is performed continuously or discontinuously in one or more frequency ranges.

20. The method as recited in one of claims 9 through 19,
wherein the radiation causes only slight heating of the liquid coating applied.

21. The method as recited in one of claims 9 through 20,
wherein the radiation causes the formation of a crystalline or amorphous coating structure of the active substance, the active substances, or the active substance mixture on the object.

22. The method as recited in one of claims 9 through 21,
wherein the object is pretreated, in particular the surface is enlarged and/or the surface is electrically charged, so that this object may absorb liquids over its entire surface area.

23. The method as recited in one of claims 9 through 22,
wherein the object to be coated or a part thereof is pretreated, in particular is cleaned of substances foreign to the object.

24. The method as recited in one of claims 9 through 23,
wherein the object or parts thereof are coated with a layer of the active substance having different or identical thicknesses.

25. The method as recited in one of claims 9 through 24,
wherein the coating thickness may lie in the nanometer range up to the millimeter range.

26. The method as recited in one of claims 9 through 25,
wherein the object is coated
using one layer or multiple layers,
using different or identical active substances,
using organic components,
in combination with components having cellular activity,
using spacings relevant for the active substance,
on at least a part of the surface,
in segments on one side or multiple sides,
on different parts of the surface using different active substances, and/or
on its entire surface.

27. The method as recited in one of claims 9 through 26,
wherein the active substance coatings may be applied with identical and/or different solubilities, and in particular include a graduated release rate of the active substance(s).

28. The method as recited in one of claims 9 through 27,
wherein the coating contains a medicinally active/pharmacologically active/pharmaceutically active component.

29. The method as recited in one of claims 9 through 28,
wherein the coating is graduated in its hardness and/or adjustable in its mechanical hardness, and in particular is a hard coating, an elastic coating, or a soft coating.

30. The method as recited in one of claims 9 through 29,
wherein the degree of hardness of the coating is determined through the energy dose, in particular using a higher dose for a harder coating and using a lower dose for a softer coating.

31. The method as recited in one of claims 9 through 30,
wherein the coating contains an identifying color.

32. The method as recited in one of claims 9 through 31,
wherein other active substances are attached onto and/or embedded into the coating.

33. The method as recited in claim 32,
wherein the other active substance is a growth factor or growth accelerator.

34. Use of the method as recited in one of claims 9 through 33 for coating
an implant, in particular a hip, knee, shoulder, or jaw implant, surgical implant, or orthopedic implant,
an onplant, in particular a surgical or orthopedic onplant,
an inlay or onlay,
a medical aid, in particular nails, screws, wires, fixators, sleeves, cannulas, needles, suture material, clamps, or drains,
a medical instrument,
a medical product, in particular invasive or active medical products,
a medical device, in particular pumps, metering devices, signaling devices, pulse generators, or insulin detectors,
a vision aid or hearing aid,
a bone replacement ceramic, in particular based on calcium and phosphate,
a bone replacement material, in particular based on ceramic, calcium and carbonate, or calcium and sulfate,
a ligament, a tendon, or a plate,
an object made of glass, ceramic, plastic, metal, glass ceramic, or metal ceramic, or
a jewelry part.

35. A product manufactured by the method as recited in one of claims 9 through 34.
